(19) United States
(12) Patent Application Publication
(10) Pub. No.: US 2023/0233492 A1
(43) Pub. Date: Jul. 27, 2023

(54) PHARMACEUTICAL COMBINATION COMPRISING A BRAIN AMINOPEPTIDASE A INHIBITOR, A DIURETIC AND A BLOCKER OF THE SYSTEMIC RENIN-ANGIOTENSIN SYSTEM

(71) Applicants: QUANTUM GENOMICS, PARIS (FR); INSTITUT NATIONAL DE LA SANTE ET DE LA RECHERCHE MEDICALE, PARIS (FR); CENTRE NATIONAL DE LA RECHERCHE SCIENTIFIQUE, PARIS (FR); COLLEGE DE FRANCE, PARIS (FR)

(72) Inventors: FABRICE BALAVOINE, PARIS (FR); CATHERINE LLORENS-CORTES, BURES SUR YVETTE (FR); YANNICK MARC, SAINT-MAUR-DES-FOSSES (FR)

(21) Appl. No.: 17/922,982

(22) PCT Filed: May 5, 2021
(86) PCT No.: PCT/EP2021/061917

§ 371 Date: Nov. 3, 2022

(30) Foreign Application Priority Data
May 6, 2020 (EP) 20305447.3

(51) Int. Cl. A61K 31/145; A61K 31/549; A61K 38/55; A61K 31/401; A61K 31/41; A61P 9/12

(52) U.S. Cl. CPC A61K 31/145; A61K 31/549; A61K 38/556; A61K 31/401; A61K 31/41; A61P 9/12

(57) ABSTRACT

The present invention relates to a pharmaceutical combination comprising (i) firibastat, (ii) a diuretic and (iii) a blocker of the systemic renin-angiotensin system selected from the group consisting of angiotensin I converting enzyme inhibitors (ACEIs) and angiotensin II receptor type 1 (AT1R) antagonists. Said composition is particularly useful for the treatment of hypertension and related diseases and conditions.

### FIELD OF THE INVENTION

The invention relates to a pharmaceutical combination comprising (i) (3S,3S′) 4,4′-disulfanediylbis(3-aminobutane 1-sulfonic acid) or a pharmaceutically acceptable salt or solvate thereof, (ii) a diuretic and (iii) a blocker of the systemic renin-angiotensin system selected from the group consisting of angiotensin I converting enzyme inhibitors (ACEIs) and angiotensin II receptor type 1 (AT1R) antagonists, and to a method useful for the treatment of hypertension and related diseases and conditions.

### TECHNICAL BACKGROUND

Arterial hypertension (HTN) is a global public health issue. According to World Health Organization statistics (World Health Organization 2013. A global brief on hypertension: Silent killer, global public health crisis. World Health Day), one out of three adults worldwide suffers from high blood pressure (BP) and prevalence of HTN is rising sharply. The number of hypertensive adults from now to 2025 could increase up to 60% and reach 1.56 billion.

HTN is one of the leading risk factors for coronary heart disease, heart failure, stroke, and renal insufficiency. It is assumed to be the cause of about half of strokes and heart diseases. Effective BP management has been shown to be the best determinant of cardiovascular risk reduction and decrease of the incidence of stroke, heart attack and heart failure. Antihypertensive medication is recommended for most adults with systolic BP 140 mm Hg or diastolic BP ≥90 mm Hg. But, even though the epidemiological association between high BP and cardiovascular morbidity and mortality is well known, and despite the fact that sufficient evidence exists to justify antihypertensive treatment, and the availability of more than 75 antihypertensive agents distributed over as many as 9 different classes, BP is often not adequately controlled. Indeed, approximately 2 out of 3 patients diagnosed with HTN do not have their BP controlled (<140/90 mmHg) (Benjamin E J, et al. Heart Disease and Stroke Statistics-2018 update: a report from the American Heart Association. Circulation. 2018; 137:e67-e492) and in low-income and middle-income countries, more than 70% of the treated patients with HTN have uncontrolled BP.

Uncontrolled HTN is even more common in obese patients, in patients of African ancestry and other minority patients, and in patients with diabetes mellitus or renal insufficiency in whom high BP is associated with low renin levels. Approximately 20% of the worldwide hypertensive population meet the criteria for apparent treatment resistant HTN (BP above goal levels despite concurrent use of adequately dosed antihypertensive drugs of 3 different classes including a diuretic, or BP below goal levels while taking at least antihypertensive drugs of 4 different classes, including a diuretic) (Carey R M, et al. Resistant hypertension: detection, evaluation, and management: A Scientific Statement from the American Heart Association. Hypertension. 2018; 72:e53-e90). Consequently, there is still an unmet medical need to develop new effective and safe classes of antihypertensive drugs acting through alternative pathways and to explore new drugs associations to further improve BP control and reduce the associated cardiovascular risk in patients.

HTN is an arterial disorder whose causes generally remain unknown. It is a multifactorial and polygenic disorder, in which various mechanisms contribute to a greater or lesser extent to increasing blood pressure. Extrinsic factors which may participate include obesity, sedentary lifestyle, excessive alcohol or salt intake, and stress. Intrinsic factors suggested to play a role include fluid retention, sympathetic nervous system activity and constriction of blood vessels. Several classes of antihypertensive agents acting on these intrinsic factors through different mechanisms of action, are widely used for the treatment of HTN and related diseases and conditions. Those classes include the thiazide diuretic agents, the beta-adrenergic blockers (“beta blockers”), the alpha/beta adrenergic blockers, the non-specific adrenergic blocking agents, the angiotensin I converting enzyme (EC 3.4.15.1) inhibitors (ACEIs), the angiotensin II receptor type 1 (AT1R) antagonists (or blockers [ARBs]), the calcium channel antagonists or blockers (CCBs), the renin inhibitors and the direct vasodilators. Each therapeutic class comprises a very large number of drugs, among them the drugs listed below which are representatives but not the only members of their classes.

The thiazide diuretics include chlorothiazide, hydrochlorothiazide (or HCTZ), chlorthalidone, indapamide, polythiazide, and hydroflumethiazide. The drugs in this class lower BP through several mechanisms. By promoting sodium loss, they lower blood volume. At the same time, the pressure of the walls of blood vessels, the peripheral vascular resistance, is lowered. Thiazide diuretics are commonly used as the first choice for reduction of mild HTN and are commonly used in combination with other antihypertensive drugs. In particular, combinations of hydrochlorothiazide, and to a less extent chlorthalidone, with specific ACEIs, ARBs, beta blockers and other diuretics, are currently available combination drugs for antihypertension.

The CCBs include amlodipine, diltiazem, felodipine, isradipine, nicardipine, nifedipine, nisoldipine, verapamil. CCBs lower BP by preventing calcium from entering the cells of heart and arteries. Calcium causes the heart and arteries to contract more strongly. By blocking calcium, calcium channel blockers allow blood vessels to relax and open. CCBs are available in short-acting and long-acting forms. Short-acting medications work quickly, but their effects last only few hours. Long-acting medications are slowly released to provide a longer lasting effect. CCBs are also commonly used in combination with other antihypertensive drugs or with cholesterol-lowering drugs such as statins. In particular, combinations of amlodipine with specific ACEIs and ARBs are currently available combination drugs for the treatment of HTN.

The ACEIs act by inhibiting the production of angiotensin II (AngII), a peptide substance that by acting on AT1 receptors both induces constriction of blood vessels and sodium retention, which leads to water retention and increased blood volume. There are many ACEIs currently available in the market, including captopril, ramipril, quinapril, enalapril, perindopril, lisinopril, fosinopril and benazepril. The primary difference between these drugs is their onset and duration of action.

The ARBs, such as losartan, candesartan, irbesartan, telmisartan, valsartan, olmesartan, eprosartan and azilsartan, block the action of AngII on AT1 receptors rather than blocking its production (like ACEIs).

ACEIs and ARBs thus target the systemic renin-angiotensin system (RAS) and more specifically AngII, either by preventing its formation through ACE inhibition or by preventing angiotensin II from binding to AT1 receptors. In both cases, inhibition leads to vasodilatation and reduction in BP.

Recent evidences support that a functional RAS, controlling cardiovascular functions and body fluid homeostasis, is also present in the brain (Llorens-Cortes C. and Mendelsohn F A. Organisation and functional role of the brain angiotensin system. J Renin Angiotensin Aldosterone Syst 2002 September; 3 Suppl 1:S39-S48). Hyperactivity of the brain RAS and particularly of aminopeptidase A (APA), a membrane-bound zinc metalloprotease involved in vivo in the conversion of brain AngII and to angiotensin III (AngIII) plays a critical role in mediating BP levels in various animal models of HTN (Marc Y. and Llorens-Cortes C. The role of the brain renin-angiotensin system in hypertension: Implications for new treatment. Prog Neurobiol. 2011 Jul. 7; 95(2):89-103). Several studies pointed out that in the brain, AngIII and not AngII as established at the periphery, contitutes one of the major effector peptides of the brain RAS in the control of BP and arginine vasopressin (AVP) release (Zini S., et al. Identification of metabolic pathways of brain angiotensin II and Ill using specific aminopeptidase inhibitors: Predominant role of angiotensin Ill in the control of vasopressin release. Proc. Natl. Acad. Sci. USA 1996 Oct. 15; 93(21):11968-73). Furthermore, brain AngIII exerts a tonic stimulatory action on the control of BP in hypertensive animals (Reaux A., at al. Aminopeptidase A inhibitors as potential central antihypertensive agents. Proc Natl Acad Sci USA. 1999 Nov. 9; 96(23):13415-20). Therefore, brain APA, the enzyme generating AngIII in the brain RAS, constitutes a relevant therapeutic target for treatment of arterial hypertension and centrally active APA inhibitors represent a new class of antihypertensive agents (Gao J. et al, A new strategy for treating hypertension by blocking the activity of the brain renin-angiotensin system with aminopeptidase A inhibitors. Clin Sci (Lond). 2014 August; 127(3):135-48).

Among these novel antihypertensive agents, one can cite in particular firibastat (also known as RB150 or QGC001) which is a prodrug of the selective aminopeptidase A (APA) inhibitor 3-amino 4-mercaptobutanesulfonic acid (also called EC33). Firibastat is chemically defined as (3S)-3-Amino-4[[(2S)-2-amino-4-sulfobutyl]disulfanyl)]butane-1-sulfonic acid or (3S,3S′) 4,4′-disulfanediylbis(3-aminobutane 1-sulfonic acid). Firibastat can be a trihydrate form as disclosed in PCT/EP2011/067524.

Oral administrations of firibastat (15 to 150 mg/kg) induce a dose-dependent decrease in BP in spontaneously hypertensive rats (SHRs), an experimental model of essential HTN (Marc Y., et al. Central antihypertensive effects of orally active aminopeptidase A inhibitors in spontaneously hypertensive rats. Hypertension. 2012 August; 60(2):411-8) and in conscious hypertensive deoxycorticosterone acetate (DOCA) salt rats, an experimental model of HTN associated with salt-sensitivity and low plasma renin levels, known to be poorly responsive to systemic RAS blockers (Bodineau L., et al, Orally active aminopeptidase A inhibitors reduce blood pressure: a new strategy for treating hypertension. Hypertension. 2008 May; 51(5):1318-25). Interestingly, firibastat was found to lower BP in DOCA-salt rats and SHRs first by decreasing vasopressin release, increasing aqueous diuresis and natriuresis, thereby decreasing blood volume and BP to control values, and secondly by lowering sympathetic tone, thereby reducing vascular resistances and consequently decreasing BP. Furthermore, monotherapy with firibastat was found to lower BP both in mild to moderate hypertensive patients (Azizi M., et al. A pilot double-blind randomized placebo-controlled crossover pharmacodynamic study of the centrally active aminopeptidase A inhibitor, firibastat, in hypertension. J Hypertens. 2019 August; 37(8):1722-1728) and in a diverse high-risk hypertensive population known to have a poor BP response to systemic RAS blockers, such as ACEis and ARBs due to high salt sensitivity, low plasma renin activity or sympathetic nervous system overactivity (Ferdinand K C., et al. Efficacy and Safety of Firibastat, A First-in-Class Brain Aminopeptidase A Inhibitor, in Hypertensive Overweight Patients of Multiple Ethnic Origins. Circulation. 2019 Jul. 9; 140(2):138-146).

The classical approach to initial pharmacological treatment of HTN has focused on monotherapy by ranking antihypertensive drugs in order of priority according to clinical parameters and patient characteristics (age, ethnic origin, presence of comorbities). However, applying this strategy in clinical practice has not been so successful, and cardiovascular disease still causes a huge amount of deaths and disabilities. The pathophysiological complexity of HTN often limits the achievement of important BP reductions with a single antihypertensive drug. Monotherapy frequently stimulates compensatory reflexes that counteract the pharmacologically induced reduction in BP. This compensation tends to hinder successful BP lowering. When hypertensive patients do not achieve adequate control of their BP, the options to try and achieve required treatment goals are to increase the dose of monotherapy (which increases the risk of side effects) or when possible to use combinations of antihypertensive drugs acting on different mechanisms which tend to lead to a more intense effect on BP. Under certain circumstances, antihypertensive drugs with different mechanisms of action have been combined to better target the underlying multiple physiologic pathways contributing to HTN. However, simply using any combination of drugs having different modes of action does not necessarily lead to combinations with advantageous. Drug classes without additive antihypertensive effects should not be combined. For instance, CCBs should not be combined with diuretics as dual therapy because both drug classes are natriuretic and cause reflex activation of the RAS. In addition, an ACEI (or ARB) should not be combined with a beta-blocker if the rationale for the combination is to improve BP control. Finally, it is not rational to combine drugs directly acting on the RAS, including ACEIs, ARBs, or renin inhibitors. While some antihypertensive combinations may have synergistic BP-lowering effects, others may have no benefit or even negative effects. For instance, the combination of two antihypertensive agents that inhibits sympathetic activity by differing pharmacologic mechanisms, the centrally-acting alpha-adrenergic agonist, clonidine, and the peripheral alpha-adrenergic antagonist, prazosin, was inappropriate in antihypertensive therapy. Thus, if a patient is treated with one of these two drugs, addition of the other does not cause a further reduction in BP decrease, but BP increases (Kapocsi J., et al. Prazosin partly blocks clonidine-induced hypotension in patients with essential hypertension. Eur J Clin Pharmacol. 1987:32(4):331-4).

Antihypertensive monotherapy normalizes BP in no more than 30-40% of patients, even those with mild to moderate HTN (stage 1 or 2), and it is not fully effective in patients with stage 3 HTN and in high-/very high-risk patients for whom rapid normalization of BP is important goal. Therefore, in their latest guidelines for management of arterial HTN, the European Society of Hypertension and the European Society of Cardiology ESH/ESC have recommended that drug treatment should be started with a combination of two antihypertensive drugs, preferentially in one pill, in all hypertensive patients, and obviously whenever patients have a high initial BP or are classified as being at high/very high cardiovascular risk due to the presence of organ damage, diabetes, or cardio renal disease (Williams B. et al., 2018 ESC/ESH Guidelines for the management of arterial hypertension. Eur Heart J. 2018 Sep. 1; 39(33):3021-3104). Similarly, the 2017 American College of Cardiology and the American Heart Association guidelines state also that initiation of antihypertensive drug therapy with 2 first-line agents of different classes, either as separate agents or in a single-pill combination, is recommended in adults with stage 2 HTN and an average BP more than 20/10 mmHg above their BP target (Whelton P K., et al. 2017 ACC/AHA/AAPA/ABC/ACPM/AGS/APhA/ASH/ASPC/NMA/PCNA Guideline for the Prevention, Detection, Evaluation, and Management of High Blood Pressure in Adults: Executive Summary: A Report of the American College of Cardiology/American Heart Association Task Force on Clinical Practice Guidelines. J Am Coll Cardiol. 2018 May 15; 71(19):2199-2269).

Besides improving BP control in treated hypertensive patients with the available armamentarium of drugs, epidemiological studies support the need of developing new combinations of alternative antihypertensive drugs which may interfere with the mechanisms involved in the genesis and maintenance of elevated BP in difficult-to-treat and/or resistant hypertensive patients, in order to reduce the associated risks of cardiovascular diseases such as myocardial infarction, cardiac arrest, stroke, or renal dysfunction.

In that context, the inventors identified a very promising combination of drugs allowing a significant hypotensive effect which could improve BP control in patients with difficult-to-treat or resistant HTN. Surprisingly, the inventors identified a combination of three drugs, (i) firibastat, a brain APA inhibitor, (ii) a diuretic and (iii) a systemic RAS blocker, exerting their antihypertensive effects through distinct and complementary mechanisms of action and allowing a significant hypotensive effect. More specifically, it was found in conscious hypertensive DOCA-salt rats, which constitute an experimental model of salt-sensitive HTN, that a combination of firibastat, hydrochorothiazide and enalapril achieves greater therapeutic effect than the administration of each of these agents alone and even than the administration of each dual drug combination (i.e. firibastat combined with enalapril, firibastat combined with hydrochorothiazide and hydrochorothiazide combined with enalapril).

### DESCRIPTION OF THE FIGURES

FIG. 1. (A) Effects of enalapril, HCTZ or firibastat, given orally alone or in combination on mean arterial blood pressure (MABP) in conscious DOCA-salt rats.

MABP values (mmHg, mean±SEM) at baseline or 5 hours after a single oral administration of saline, enalapril (10 mg/kg), HCTZ (10 mg/kg), firibastat (30 mg/kg), firibastat (30 mg/kg) plus enalapril (10 mg/kg), firibastat (30 mg/kg) plus HCTZ (10 mg/kg), enalapril (5 mg/kg) plus HCTZ (5 mg/kg), firibastat (30 mg/kg) plus enalapril (5 mg/kg) plus HCTZ (5 mg/kg) in conscious DOCA-salt rats. (n=6 for each treatment). One-way ANOVA followed by Sidak's multiple comparisons test, ns: non-significant, * P<0.05; ** P<0.01; *** P<0.0001, when compared to corresponding baseline MABP values.

FIG. 1. (B) Changes from baseline after 5 hours post-dosing in mean arterial blood pressure (MABP) after a single oral administration of enalapril, HCTZ or firibastat given alone or in combination in conscious DOCA-salt rats.

Mean±SEM changes in MABP (mmHg) from baseline after 5 hours following a single oral administration of saline, enalapril (10 mg/kg), HCTZ (10 mg/kg), firibastat (30 mg/kg), firibastat (30 mg/kg) plus enalapril (10 mg/kg), firibastat (30 mg/kg) plus HCTZ (10 mg/kg), enalapril (5 mg/kg) plus HCTZ (5 mg/kg), firibastat (30 mg/kg) plus enalapril (5 mg/kg) plus HCTZ (5 mg/kg) in conscious DOCA-salt rats (n=6 per group). One-way ANOVA followed by Sidak's multiple comparisons when compared to changes in MABP values obtained in DOCA-salt rats receiving saline, * P<0.01; ** P<0.001; *** P<0.0001 or Tukey's multiple comparisons test when compared to changes MABP values obtained in DOCA-salt receiving firibastat plus enalapril plus HCTZ, #P<0.05; ##P<0.01; ###P<0.0001.

FIG. 2. Effects of enalapril, HCTZ or firibastat, given orally alone or in combination on heart rate (HR) in conscious DOCA-salt rats.

HR values (bpm, mean±SEM) at baseline or 5 hours after a single oral administration of saline, enalapril (10 mg/kg), HCTZ (10 mg/kg), firibastat (30 mg/kg), firibastat (30 mg/kg) plus enalapril (10 mg/kg), firibastat (30 mg/kg) plus HCTZ (10 mg/kg), enalapril (5 mg/kg) plus HCTZ (5 mg/kg), firibastat (30 mg/kg) plus enalapril (5 mg/kg) plus HCTZ (5 mg/kg) in conscious DOCA-salt rats. (n=6 for each treatment). One-way ANOVA followed by Sidak's multiple comparisons test, ns: non-significant, when compared to corresponding baseline HR values.

FIG. 3. (A) Time course of mean arterial blood pressure (MABP) after chronic oral administration of enalapril, HCTZ or firibastat, given orally in combination in conscious DOCA-salt rats.

MABP values (mmHg, mean±SEM) on day 8 at baseline, 5, 9 or 24 hours after a daily chronic oral administration of saline, enalapril (5 mg/kg) plus HCTZ (5 mg/kg) or firibastat (30 mg/kg) plus enalapril (5 mg/kg) plus HCTZ (5 mg/kg) over 8 consecutive days in conscious DOCA-salt rats. (n=12 for each treatment). One-way ANOVA followed by Holm-Sidak's multiple comparisons test, * P<0.05; ** P<0.01; *** P<0.0001, when compared to the corresponding studied time MABP values obtained in DOCA-salt rats receiving saline.

FIG. 3. (B) Time course of mean arterial blood pressure (MABP) changes from baseline after a daily chronic oral administration of enalapril, HCTZ or firibastat given in combination during 8 days in conscious DOCA-salt rats. Mean±SEM changes in MABP (mmHg) from baseline to day 8 after 5, 9 or 24 hours following a daily chronic oral administration of saline, enalapril (5 mg/kg) plus HCTZ (5 mg/kg) or firibastat (30 mg/kg) plus enalapril (5 mg/kg) plus HCTZ (5 mg/kg) during 8 consecutive days in conscious DOCA-salt rats. (n=12 for each treatment). One-way ANOVA followed by Holm-Sidak's multiple comparisons test, * P<0.05; ** P<0.001; *** P<0.0001 when compared to changes in MABP values obtained in DOCA-salt rats receiving saline or Sidak's multiple comparisons test, #P<0.05, when compared to changes in MABP values obtained in DOCA-salt receiving firibastat plus enalapril plus HCTZ.

FIG. 4. Time course of heart rate (HR) after chronic oral administration of enalapril, HCTZ or firibastat, given in combination in conscious DOCA-salt rats.

HR values (bpm, mean±SEM) 5, 9 or 24 hours after a daily chronic oral administration of saline, enalapril (5 mg/kg) plus HCTZ (5 mg/kg) or firibastat (30 mg/kg) plus enalapril (5 mg/kg) plus HCTZ (5 mg/kg) during 8 days in conscious DOCA-salt rats. (n=12 for each treatment). One-way ANOVA followed by Tukey's multiple comparisons test, ns: non-significant, when compared to DOCA-salt rats receiving saline or enalapril plus HCTZ.

FIG. 5. Effects of chronic oral RB150 treatment on plasma arginine vasopressin (AVP) release in conscious hypertensive DOCA-salt rats.

Plasma AVP levels were assessed by radioimmunoassay after daily 10-day chronic oral administration of saline, enalapril (5 mg/kg) plus HCTZ (5 mg/kg) or firibastat (30 mg/kg) plus enalapril (5 mg/kg) plus HCTZ (5 mg/kg) in conscious DOCA-salt rats. The results were expressed as picogram of AVP per milliliter of plasma. Values are expressed as mean±SEM of 10 animals individually analyzed for each condition. One-way ANOVA followed by Tukey's test, ns: non-significant, * P<0.05; ** P<0.001 when compared to the corresponding plasma AVP values obtained in DOCA-salt rats receiving saline or enalapril plus HCTZ.

### SUMMARY OF THE INVENTION

In one embodiment, the present invention relates to a pharmaceutical combination comprising (i) firibastat, (ii) a diuretic and (iii) a third active ingredient selected from the group consisting of ACEIs and ARBs.

Said combination is particularly useful for the treatment of arterial HTN or indirectly or directly related diseases.

In accordance with another embodiment of the present invention, a method is disclosed for the treatment of HTN and indirectly or directly related diseases. The method and use of the invention comprises administering to a subject in need of such treatment an effective amount of a pharmaceutical composition comprising (i) firibastat, (ii) a diuretic and (iii) a third active ingredient selected from the group consisting of ACEIs and ARBs, or, where appropriate, for each active ingredient (i)-(iii) a pharmaceutically acceptable salt or solvate thereof.

In yet another embodiment, the invention relates to a kit of parts comprising a pharmaceutical combination as defined above, for a simultaneous or sequential administration, preferably for simultaneous administration.

### DESCRIPTION OF THE INVENTION

The present invention relates to a pharmaceutical combination, comprising (i) firibastat, (ii) a diuretic and (iii) a third active ingredient selected from the group consisting of ACEIs and ARBs, more particularly for use in the treatment of arterial HTN or indirectly or directly related diseases.

The invention likewise relates to the use of (i) firibastat, (ii) a diuretic and (iii) a third active ingredient selected from the group consisting of ACEIs and ARBs, for the manufacture of a medicament for the treatment of arterial HTN or indirectly or directly related diseases.

The invention likewise relates to a method for the treatment of arterial HTN or indirectly or directly related diseases, comprising administering to a patient, including human and non-human subjects, a therapeutically effective amount of (i) firibastat, (ii) a diuretic and (iii) a third active ingredient selected from the group consisting of ACEIs and ARBs.

The invention furthermore relates to a kit of parts comprising (i) firibastat or a pharmaceutically acceptable salt or solvate thereof, (ii) a diuretic or a pharmaceutically acceptable salt or solvate thereof, (iii) a third active ingredient selected from the group consisting of ACEIs and ARBs, or a pharmaceutically acceptable salt or solvate thereof, for a simultaneous or sequential administration of said three active ingredients (i)-(iii), more preferably in the form of one or more separate dosage units of the active ingredients (i) to (iii) or pharmaceutical compositions comprising the active ingredients (i) to (iii).

According to the invention, the term “comprise(s)” or “comprising” (and other comparable terms, e.g., “containing,” and “including”) is “open-ended” and can be generally interpreted such that all of the specifically mentioned features and any optional, additional and unspecified features are included. According to specific embodiments, it can also be interpreted as the phrase “consisting essentially of” where the specified features and any optional, additional and unspecified features that do not materially affect the basic and novel characteristic(s) of the claimed invention are included or the phrase “consisting of” where only the specified features are included, unless otherwise stated.

According to the invention, the diuretics include more particularly chlorothiazide, hydrochlorothiazide, chlorthalidone, indapamide, furosemide, torsemide, amiloride, triamterene, spironolactone and eplerenone. According to a preferred embodiment, the diuretic is selected from the group consisting of hydrocholorothiazide, chorthalidone, indapamide, and amiloride. More specifically, the diuretic is hydrocholorothiazide.

According to the invention, the ACEIs include more particularly lisinopril, enalapril, quinapril, ramipril, benazepril, captopril, cilazapril, fosinopril, imidapril, moexipril, trandolapril, or perindopril. According to a preferred embodiment, the ACEI is selected from the group consisting of enalapril, perindopril, ramipril, lisinopril and benazepril. More specifically, the ACEI is enalapril.

According to the invention, the ARBs include more particularly losartan, candesartan, irbesartan, telmisartan, valsartan, olmesartan, eprosartan and azilsartan. According to a preferred embodiment, the ARB is selected from the group consisting of losartan, candesartan, valsartan, olmesartan and azilsartan. More specifically, the ARB is valsartan.

Whenever appropriate, each of the active ingredients as identified herein also encompasses a pharmaceutically acceptable salt or solvate thereof.

Firibastat is chemically defined as (3S)-3-Amino-4[[(2S)-2-amino-4-sulfobutyl]disulfanyl)]butane-1-sulfonic acid or also named (3S,3S′) 4,4′-disulfanediylbis(3-aminobutane 1-sulfonic acid). All those terms can thus be used herein interchangeably, and include zwitterionic form, pharmaceutically acceptable salt or solvate thereof, including a hydrate form. Firibastat can be a trihydrate form as disclosed in PCT/EP2011/067524. The term “firibastat” refers herein to (3S,3S′) 4,4′-disulfanediylbis(3-aminobutane 1-sulfonic acid), a zwitterionic form, pharmaceutically acceptable salt or solvate thereof, including a hydrate form.

Firibastat can be referred as a homodimer of the selective aminopeptidase A (APA) inhibitor 3-amino 4-mercaptobutanesulfonic acid (also called EC33), generated by creating a disulfide bond between thiol groups of two 3-amino 4-mercaptobutanesulfonic acid molecules. Dimerisation affords a molecule more amenable to cross the gastro-intestinal and blood-brain barriers as a prodrug. On entry into the brain, firibastat is cleaved by brain reductases to generate two active molecules of EC33, which inhibit brain APA activity, block brain AngIII formation, and decrease BP.

(3S,3S′) 4,4′-disulfanediylbis(3-aminobutane 1-sulfonic acid) and use thereof as anti-hypertensive agent have been disclosed in the patent application WO2004/007441. The antihypertensive effects of firibastat treatment (500 mg twice a day) in mild to moderate hypertensive patients and in a diverse high-risk hypertensive population have been confirmed already (Azizi M., et al. A pilot double-blind randomized placebo-controlled crossover pharmacodynamic study of the centrally active aminopeptidase A inhibitor, firibastat, in hypertension. J Hypertens. 2019 August; 37(8):1722-1728; Ferdinand K C., et al. Efficacy and Safety of Firibastat, A First-in-Class Brain Aminopeptidase A Inhibitor, in Hypertensive Overweight Patients of Multiple Ethnic Origins. Circulation. 2019 Jul. 9; 140(2):138-146).

As mentioned above, references hereinafter to (3S,3S′) 4,4′-disulfanediylbis(3-aminobutane 1-sulfonic acid) or firibastat include the zwitterionic form and its pharmaceutically acceptable salts and solvates.

The person skilled in the art will recognize that firibastat may contain at least one positive and one negative charge so that firibastat includes zwitterionic forms thereof. In chemistry, a zwitterion (also called an inner salt), is a molecule with two or more functional groups, of which at least one has a positive and one has a negative electrical charge and the charges on the different functional groups balance each other out, and the molecule as a whole is electrically neutral.

The specialist in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the components (or active ingredients) (i)-(iii) are within the scope of the present invention. Solvates of (3S,3S′) 4,4′-disulfanediylbis(3-aminobutane 1-sulfonic acid) are within the scope of the present invention. Organic compounds can exist in more than one crystalline form. For example, crystalline form may vary from solvate to solvate. Thus, all crystalline forms of (3S,3S′) 4,4′-disulfanediylbis(3-aminobutane 1-sulfonic acid) or the pharmaceutically acceptable solvates thereof are within the scope of the present invention.

It will also be appreciated by the person skilled in the art that (3S,3S′) 4,4′-disulfanediylbis(3-aminobutane 1-sulfonic acid) may also be utilized in the form of pharmaceutically acceptable salts thereof. The pharmaceutically acceptable salts of (3S,3S′) 4,4′-disulfanediylbis(3-aminobutane 1-sulfonic acid) include conventional salts formed from pharmaceutically acceptable inorganic or organic acids or bases as well as quaternary ammonium salts and aminoacids. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic etc. Other acids such as oxalic, while not in themselves pharmaceutically acceptable, may be useful in the preparation of salts useful as intermediates in obtaining the compounds of the present invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. More specific examples of suitable aminoacid salts include L- and D-forms of tryptophan, serine, cystine, valine, arginine, glycine, arginine, or lysine. A crystalline form of (3S,3S′) 4,4′-disulfanediylbis(3-aminobutane 1-sulfonic acid) with L-lysine and a process for the preparation of this crystalline form are disclosed in PCT/EP2013/072028.

In preferred embodiments, the indirectly or directly diseases related to HTN are selected from the group consisting of diseases of the heart, the peripheral and cerebral vascular system, the brain, the eye and the kidney. In particular, diseases include primary and secondary arterial HTN, ictus, myocardial ischaemia, heart failure, renal failure, myocardial infarction, peripheral vascular disease, diabetic proteinuria, Syndrome X or glaucoma. It may also include more particularly nephropathy, retinopathy or neuropathy in hypertensive diabetic patients. According to a particular embodiment, the indirectly or directly disease is heart failure.

Within the context of the invention, the term treatment denotes curative, symptomatic, and preventive treatment. Combinations or compositions of the invention can be used in subjects with existing HTN. The combination or the compositions of the invention will not necessarily cure the patient who has HTN but will control BP in a satisfactory manner, delaying or slowing thereby the progression or preventing thereby further complications of HTN, such as the directly or indirectly diseases as mentioned above. This will ameliorate consequently the patients' condition. The combination or the compositions of the invention can also be administered to those who do not have indirectly or directly diseases yet but who would normally develop the diseases or be at increased risk for said diseases, so that they will not develop said diseases. Treatment thus also includes delaying the development of indirectly or directly diseases in an individual who will ultimately develop said diseases or would be at risk for the diseases due to age, familial history, genetic or chromosomal abnormalities. By delaying the onset of the indirectly or directly diseases, compositions of the invention have prevented the individual from getting the diseases during the period in which the individual would normally have gotten the diseases or reduce the rate of development of the diseases or some of its effects but for the administration of compositions of the invention up to the time the individual ultimately gets the diseases.

The terms “patient,” “subject,” “individual,” and the like are used interchangeably herein, and refer to any human or non-human mammalian subject, including humans, laboratory, domestic, wild or farm animals. In certain non-limiting embodiments, the patient, subject or individual is a human. In other embodiments, the patient, subject or individual is a domestic animal, such as feline or canine subjects, a farm animal, such as but not limited to bovine, equine, caprine, ovine, and porcine subjects, wild animals (whether in the wild or in a zoological garden), research animals, such as mice, rats, rabbits, goats, sheep, pigs, dogs, cats, and the like, avian species, such as chickens, turkeys, songbirds, and the like, i.e., for veterinary medical use. Preferably the subject is a human patient whatever its sex (women or men) or age, generally an adult.

Surprisingly, the inventors identified a combination of at least three drugs, (i) firibastat, a brain APA inhibitor, (ii) a diuretic and (iii) a systemic RAS blocker, exerting their antihypertensive effects through distinct and complementary mechanisms of action and allowing a significant hypotensive effect. The unexpected advantage of this combination is illustrated by the potentiated BP lowering effect and the improved benefit observed over the dual combinations of each drug. The combination comprising (i) firibastat, (ii) a diuretic and (iii) a systemic RAS blocker, represents a very promising therapy to improve BP control, in patients with HTN and in particular in patients with difficult-to-treat HTN, and more specifically hypertensive patients not adequately controlled by a dual therapy, such as a diuretic and a systemic RAS blocker. More specifically, the combination comprising (i) firibastat, (ii) a diuretic and (iii) a systemic RAS blocker constitutes an alternative or adjunct therapy for hypertensive patients, and more specifically for high-risk for hypertensive patients, including those with salt-sensitivity, low plasma renin activity or sympathetic nervous system overactivity. Such patients are known to be associated with poor response to antihypertensive treatment with diuretics and/or systemic RAS blockers, used separately as single drug therapy or combined in a dual therapy.

In one embodiment, the present invention relates to a pharmaceutical combination, comprising (i) firibastat, (ii) a diuretic and (iii) a third active ingredient selected from the group of systemic RAS blockers consisting of ACEIs and ARBs.

In a preferred embodiment, the diuretic is selected from the group consisting of hydrochlorothiazide, indapamide, furosemide and chlorthalidone. In a more preferred embodiment, the diuretic is hydrochlorothiazide.

In another preferred embodiment, the systemic RAS blocker is selected from the group of ACEIs consisting of enalapril, perindopril, ramipril and benazepril, or from the group of ARBs consisting of losartan, valsartan, candesartan and azilsartan. In a more preferred embodiment, the systemic RAS blocker is enalapril or valsartan.

In one embodiment, the present invention relates to a pharmaceutical combination, comprising (i) firibastat, (ii) hydrochlorothiazide and (iii) enalapril.

According to an embodiment of the invention, (i) firibastat, (ii) diuretic, and a systemic RAS blocker are administered simultaneously or sequentially, in the form of separate pharmaceutical compositions, each pharmaceutical composition comprising one of active ingredients (i)-(iii) in a pharmaceutically acceptable vehicle. In another embodiment, (i) firibastat, (ii) diuretic, and a systemic RAS blocker are administered simultaneously or sequentially, in the form of two separate pharmaceutical compositions, one pharmaceutical composition comprising one of said active ingredients selected from components (i)-(iii), and the other pharmaceutical composition comprising the other two of said active ingredients selected from components (i)-(iii), each pharmaceutical composition further comprising a pharmaceutically acceptable vehicle. In another embodiment, (i) firibastat, (ii) a diuretic, and a systemic RAS blocker are administered simultaneously in the form of a single pharmaceutical composition, said pharmaceutical composition further comprising a pharmaceutically acceptable vehicle. In the context of the present invention, the terms “pharmaceutical combination” refer to one or the other of these aspects.

According to the embodiment where (i) firibastat, (ii) diuretic, and a systemic RAS blocker are administered simultaneously or sequentially, in the form of two separate pharmaceutical compositions, the pharmaceutical composition comprising one of said active ingredients is preferably a pharmaceutical composition comprising firibastat, and the second pharmaceutical composition comprising the other two of said active ingredients selected from components (i)-(iii) is a pharmaceutical composition comprising the active ingredients (ii) and (iii), each of said pharmaceutical compositions further comprising a pharmaceutically acceptable vehicle.

The pharmaceutical combination or composition(s) according to the present invention is (or are) useful in the treatment of HTN or indirectly or directly related diseases. In treating the arterial HTN, preferred dosages for the active ingredients of the pharmaceutical combination according to the present invention are therapeutically effective dosages, especially those which are commercially available.

The pharmaceutical compositions of the invention as described above advantageously contain one or more supports or vehicles that are pharmaceutically acceptable. The term “pharmaceutically acceptable support” refers to carrier, adjuvant, or excipient acceptable to the subject from a pharmacological/toxicological point of view and to the manufacturing pharmaceutical chemist from a physical/chemical point of view regarding to composition, formulation, stability, subject acceptance and bioavailability.

More preferably, the composition(s) is (are) intended for oral administration, the pharmaceutically acceptable support or vehicle is thus suitable for an oral administration. As examples, mention may be made of saline, physiological, isotonic, buffered solutions, etc. compatible with pharmaceutical use and known to persons skilled in the art.

The pharmaceutical composition(s), as described above, can be prepared by mixing the three active ingredients, either all together or each one or two independently with a physiologically acceptable support, an excipient, a binder, a diluent, etc. The pharmaceutical composition(s) of the invention is (are) more specifically for a simultaneous sequential administration, preferably for simultaneous administration, of the three active ingredients (i)-(iii).

The pharmaceutical composition(s) is (are) then administered orally or non-orally, for instance via the parenteral, intravenous, cutaneous, nasal, rectal route or via aerosol delivery to the lungs. If the active ingredients are formulated independently, the corresponding formulations can be mixed together extemporaneously using a diluent and are then administered or can be administered independently of each other, either successively or sequentially.

Preferably, the composition(s) of the invention is (are) administered orally.

The pharmaceutical compositions of the invention include formulations, such as granules, powders, tablets, gel capsules, syrups, emulsions and suspensions, and also forms used for non-oral administration, for instance injections, sprays or suppositories.

The pharmaceutical forms can be prepared via the known conventional techniques.

The preparation of an orally administered solid pharmaceutical form will be performed by the following process: an excipient (for example lactose, sucrose, starch, mannitol, etc.), a disintegrant (for example calcium carbonate, calcium carboxymethylcellulose, etc.), a binder (for example starch, gum arabic, carboxymethylcellulose, polyvinylpyrrolidone, hydroxypropylcellulose, etc.) and a lubricant (for example talc, magnesium stearate, etc.) are, for example, added to the active ingredient(s) and the mixture obtained is then tabletted. If necessary, the tablet can be coated via the known techniques, in order to mask the taste (for example with cocoa powder, mint, etc.) or to allow enteric dissolution or sustained release of the active ingredients. Pharmaceutically acceptable colorants may be added. Pharmaceutical forms, such as tablets, powders, sachets and gel capsules can be used for an oral administration.

The liquid pharmaceutical forms for oral administration include solutions, suspensions and emulsions. The aqueous solutions can be obtained by dis-solving the active ingredient(s) in water, followed by addition of flavourings, colorants, stabilisers and thickener, if necessary. In order to improve the solubility, it is possible to add ethanol, propylene glycol or other pharmaceutically acceptable non-aqueous solvents. The aqueous suspensions for oral use can be obtained by dispersing the finely divided active ingredient(s) in water with a viscous product, such as natural or synthetic gums, resins, methylcellulose or sodium carboxymethylcellulose.

The pharmaceutical forms for injection can be obtained, for example, by the following process. The active ingredient(s) is (are) dissolved, suspended or emulsified either in an aqueous medium (for example distilled water, physiological saline, Ringer's solution, etc.) or in an oily medium (for example a plant oil, such as olive oil, sesameseed oil, cottonseed oil, corn oil, etc., or propylene glycol), with a dispersant, a preserving agent, an isotonicity agent and also other additives, such as, if desired, a solubilising agent or a stabiliser.

A pharmaceutical form for external use can be obtained from a solid, semi-solid or liquid composition containing the active ingredients. For example, to obtain a solid form, the active ingredients are treated, alone or as mixtures, with excipients and a thickener so as to convert them into powder. The liquid pharmaceutical compositions are prepared in substantially the same way as the forms for injection, as indicated previously. The semi-solid pharmaceutical forms are preferably in the form of aqueous or oily gels or in the form of pomade. These compositions may optionally contain a pH regulator and also other additives.

A therapeutically effective amount (i.e., an effective dosage) of a composition or of active ingredients of the invention is determined by one skilled in the art. More specifically, an effective amount is an amount that allows decreasing and maintaining BP as to control BP, in particular BP goal of <140/90 mmHg is recommended.

It will be appreciated that the amount of the active ingredients of the present invention required for use in treatment will vary with the nature of the condition being treated and the age and the condition of the subject and will be ultimately at the discretion of the attendant physician or veterinarian. In general, however, doses employed for adult human treatment will typically be in the range of 50 mg to 1500 mg per day or every other day, of firibastat. With respect to the second active ingredient, a diuretic, and to the third active ingredient selected from the group of systemic RAS blockers consisting of ACEIs and ARBs, doses employed for treatment will take into account the recommended dosages thereof.

According to a particular embodiment, the pharmaceutical combination of the invention comprises an amount of firibastat between 100 mg and 400 mg (e.g. 250 mg), an amount of hydrochorothiazide between 5 mg and 15 mg (e.g. 6.25 mg or 12.5 mg), and an amount of enalapril between 2.5 mg and 15 mg (e.g. 5 mg or 10 mg), either in one, two or three separate pharmaceutical composition(s).

According to another particular embodiment, the pharmaceutical combination of the invention comprises an amount of firibastat between 300 mg and 600 mg (e.g. 500 mg), an amount of hydrochorothiazide between 5 mg and 15 mg (e.g. 6.25 mg or 12.5 mg), and an amount of enalapril between 2.5 mg and 15 mg (e.g. 5 mg or 10 mg), either in one, two or three separate pharmaceutical composition(s).

According to another particular embodiment, the pharmaceutical combination comprises an amount of firibastat between 700 mg and 1200 mg (e.g. 1000 mg), an amount of hydrochorothiazide between 10 mg and 30 mg (e.g. 12.5 mg or 25 mg), and an amount of enalapril between 10 mg and 50 mg (e.g. 20 mg or 40 mg), either in one, two or three separate pharmaceutical composition(s).

The desired dose may conveniently be presented in a single dosage unit or several divided dosage units administered at appropriate intervals, for example as two, three, four or more sub-doses per day or every other day. The composition(s) according to the present invention may contain between 0.1-99% by weight of each active ingredient, conveniently from 30-95% by weight for tablets and capsules and 3-50% by weight for liquid preparations, the % are expressed with respect to the total amount of the said compositions. The frequency of administration of the active ingredients of the invention is between one and two administrations per day or every other day.

The relative proportions of the active ingredients of the pharmaceutical combination may vary upon the subject condition and also upon selected diuretic and systemic RAS blocker. For example, the weight ratio of firibastat relative to either hydrocholorthiazide or enalapril may range from 10/1 to 300/1 and preferably from 25/1 to 200/1.

The pharmaceutical combination can be included in a container, pack, or dispenser, also called a kit, together with instructions for administration. Corresponding instructions are given at the package insert concerning the combined administration of the respective active ingredients (i)-(iii) or pharmaceutical composition(s) comprising said active ingredients.

The present invention thus relates to kits that are suitable for the treatment by the methods or uses described above. These kits comprise a pharmaceutical combination, as defined above, containing (i) firibastat, (ii) a diuretic and (iii) a third active ingredient selected from the group of systemic RAS blockers consisting of ACEIs and ARBs, for a simultaneous or sequential administration, preferably for simultaneous administration. More particularly, the kit comprises one or more (such as two or three) separate (either single or divided) dosage units of the active ingredients (i) to (iii) or of the pharmaceutical compositions comprising the active ingredients (i) to (iii), as defined above.

According to a particular embodiment, the kit of parts comprises a pharmaceutical combination, wherein (ii) the diuretic is selected from the group consisting of hydrochlorothiazide, indapamide, amiloride and chlorthalidone, and (iii) the blocker of the systemic renin-angiotensin system is selected from the group consisting of angiotensin I converting enzyme inhibitors consisting of enalapril, perindopril, ramipril and benazepril or from angiotensin II receptor type 1 antagonists the group consisting of losartan, valsartan, candesartan, irbesartan and azilsartan.

According to a more particular embodiment, the kit of the invention comprises a pharmaceutical combination, wherein (ii) the diuretic is hydrochlorothiazide and (iii) the blocker of the systemic renin-angiotensin system is enalapril.

According to another particular embodiment, the kit of the invention comprises a pharmaceutical combination, wherein (ii) the diuretic is hydrochlorothiazide and (iii) the blocker of the systemic renin-angiotensin system is valsartan.

According to another particular embodiment, the kit of parts comprises a pharmaceutical combination, wherein (ii) the diuretic is indapamide and (iii) the blocker of the systemic renin-angiotensin system is perindopril.

According to a further particular embodiment, the kit of parts comprises a pharmaceutical combination, wherein (ii) the diuretic is chlorthalidone and (iii) the blocker of the systemic renin-angiotensin system is azilsartan.

The separate dosage units of the kit are preferably made available together in one pack and either mixed prior to administration or sequentially administered.

For simultaneous administration as fixed composition (i.e. determined amounts and specific weight ratios between the said three active ingredients), a single pharmaceutical formulation may also be prepared which includes all three active ingredients (i)-(iii).

This invention is also directed to the use of (i) firibastat, (ii) a diuretic and (iii) a third active ingredient selected from the group consisting of ACEIs and ARBs, as defined above, in the manufacture of a medicine or one, two or three pharmaceutical composition, as defined above, intended for the treatment of arterial HTN or indirectly or directly related diseases.

The terms “simultaneous or sequential administration” of active ingredients to the same subject or patient, can be carried out over a period that may be up to 2 hours or even up to 6 hours. For example, the terms include (1) a simultaneous administration of the three active ingredients (i.e. the administration of all three active ingredients is carried out within a period of less than 10 minutes, e.g. from 30 seconds to 5 minutes long), (2) an administration of the three active ingredients separately but within a period of 3 hours, and (3) an administration of each of the three active ingredients separately every hour. According to a preferred embodiment, active ingredients are simultaneously co-administered according to (1).

The examples below of compositions according to the invention are given as non-limiting illustrations.

### Examples

The amounts are expressed on a weight basis, unless otherwise stated.

### Materials and Methods

### Drugs

Firibastat was synthesized by PCAS (Limay, France). The angiotensin converting inhibitor (ACEI), enalapril was purchased from Sequoia Research (Pangbourne, United Kingdom). The diuretic, hydrochlorothiazide (HCTZ) was purchased from Sigma-Aldrich ((Saint-Louis, United-States).

The drugs were dissolved in sterile saline for in vivo per os by gavage administration.

### Animals

Male deoxycorticosterone acetate (DOCA)-salt rats weighing 250 to 350 g were purchased from Charles River Laboratories (L'Arbresle, France). Animals were randomly assigned in each group with allocation concealment, and blinding procedures were used with coding systemAnimal care and surgical procedures were performed according to the Directive 2010/63/EU. The project was submitted to the appropriate ethics committee and authorization was obtained: 30 CEEA No 59, reference number 01962.01. Animals were kept under artificial light (12 h light/12 h dark cycle) with ad libitum access to a standard diet and saline water (0.9% NaCl, 0.2% KCl).

### Surgical Methods and Mean Arterial BP Recording

Male DOCA-salt rats were anesthetized with 3% isoflurane (Iso-Vet®, Piramal, UK) for induction and 1.5-2% isoflurane for maintenance. A catheter (0.011″X.024″X.0065″) was inserted into the right femoral artery to monitor mean arterial blood pressure (MABP) and heart rate (HR). The catheters were tunneled subcutaneously to exit from the neck. Animals were allowed to recover from surgery for at least 48 h before MABP recording. Baseline MABP was recorded before drug administration during 30 minutes to 1 hour. One hour after the start of BP recording, firibastat (30 mg/kg), enalapril (5 or 10 mg/kg) and HCTZ (5 or 10 mg/kg) alone or in combination at different doses were administered to the conscious unrestrained rats by oral route. After compound administration, BP was monitored for 6 hours. MABP was calculated with the MatLab system (Phymep, Paris, France), consisting of a MatLab hardware unit and CHART software, running on a Macintosh computer. MABP and HR measurements were calculated by the BP signal.

For the chronic treatments, three groups of DOCA-salt rats were used. Saline, enalapril (5 mg/kg) plus HCTZ (5 mg/kg) or firibastat (30 mg/kg) plus enalapril (5 mg/kg) plus HCTZ (5 mg/kg) were administered orally by gavage every day. On day 8, a catheter was inserted into the right femoral artery to monitor mean arterial BP (MABP) as previously described. The animals were allowed to recover for at least 24 hours. On day 9, baseline MABP was recorded 30 minutes to 1 hour before drugs administration. Saline, enalapril (5 mg/kg) plus HCTZ (5 mg/kg) or firibastat (30 mg/kg) plus enalapril (5 mg/kg) plus HCTZ (5 mg/kg) were administered orally by gavage. MABP was then recorded at different time points after drug administration (5, 9 and 24 hours). For each time, recording period lasted 1 hour.

### Plasma Collection for AVP Levels and Electrolytes Measurement

Rats were sacrificed by decapitation 5 hours after treatment (saline, enalapril (5 mg/kg) plus HCTZ (5 mg/kg) or firibastat (30 mg/kg) plus enalapril (5 mg/kg) plus HCTZ (5 mg/kg) on day 10. Trunk blood (6-7 mL) was collected into chilled tubes containing 0.05 mL of 0.3M EDTA (pH 7.4) per mL of blood or 50 Units of heparin lithium per mL of blood on ice and centrifuged at 5000 rpm at 4° C. for 15 min.

### AVP Radioimmunoassay

Plasma was acidified by adding 0.2 volumes of 3 M HCl and was stored at −80° C. until the AVP assay. Samples were thawed on ice and then centrifuged at 20,000×g for 20 min at 4° C. AVP was extracted from plasma by mixing 0.5 mL of the supernatant with 0.5 mL of 1% trifluoroacetic acid (TFA) and loading onto a Sep-Pak C18 cartridge (Waters, Mass., USA) previously washed with 2 mL 100% acetonitrile and equilibrated with 5 mL 1% TFA. The column was then washed with 3 mL of 1% TFA and AVP was eluted with 1.5 mL of 100% acetonitrile. The samples were lyophilized and dissolved in 0.35 mL of RIA buffer (19 mM NaH₂PO₄·H₂O, 81 mM Na₂HPO₄·2H₂O, 50 mM NaCl, 0.1% TritonX-100, 0.01% NaN₃, 0.1% BSA). Plasma AVP levels were determined by radioimmunoassay (RIA) with 0.1 mL of plasma, using 0.1 mL of a polyclonal rabbit antiserum specific for AVP-[Arg8] (Peninsula Laboratories International, San Carlo, Calif., USA) displaying no cross reactivity with oxytocin at a dilution of 2:3, and 0.1 mL of [1251]-(Tyr2Arg8)-AVP 2000 Ci/mmol (PerkinElmer, Waltham, Mass., USA) as a tracer at 15,000 dpm, with incubation overnight at 4° C. We added 0.1 mL goat anti-rabbit IgG serum and 0.1 mL normal rabbit serum from Peninsula Laboratories International (San Carlo, Calif., USA) and incubated the resulting mixture for 2 h at room temperature. We then added 0.5 mL RIA buffer and centrifuged the tubes at 2,600×gat 4° C. for 20 minutes. The supernatant was removed, and the radioactivity of the precipitates was measured. The limit of detection of the AVP RIA was 0.2 pg per tube.

### Plasma Electrolytes Measurement

Plasma sodium and potassium concentrations were determined with an electrolyte analyzer from Caretium Medical Instruments Co. (Shenzhen, China).

### Statistical Analysis

Quantitative data are shown as means±SEM. Normality was assessed with the d'Agostino-Pearson test. ANOVA was performed after verification that the residuals were normally distributed. If normality was confirmed, comparisons between multiple groups were performed by one-way ANOVA, followed by Tukey, Holm-Sidak or Sidak's test for multiple comparisons. Differences were considered significant if the P value was <0.05. Statistical analyses were performed with Prism software (GraphPad Software).

### RESULTS

Effects of acute oral administration of firibastat, enalapril and HCTZ alone or in combination on BP and HR in freely moving DOCA-salt rats.

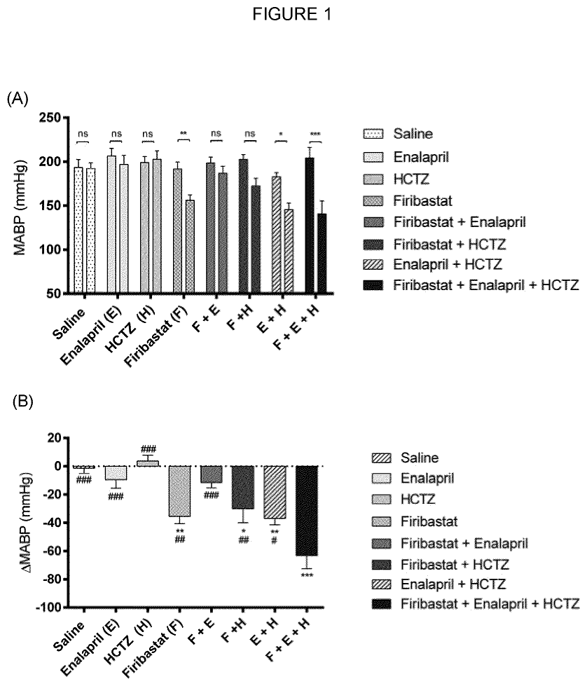

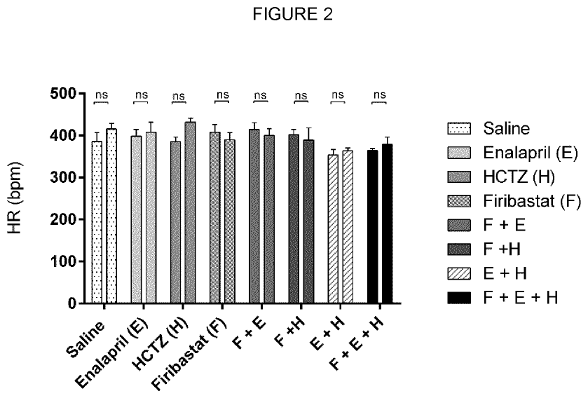

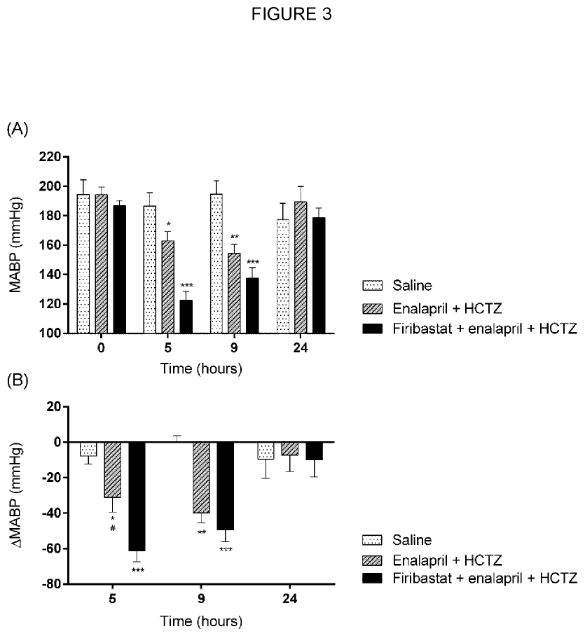

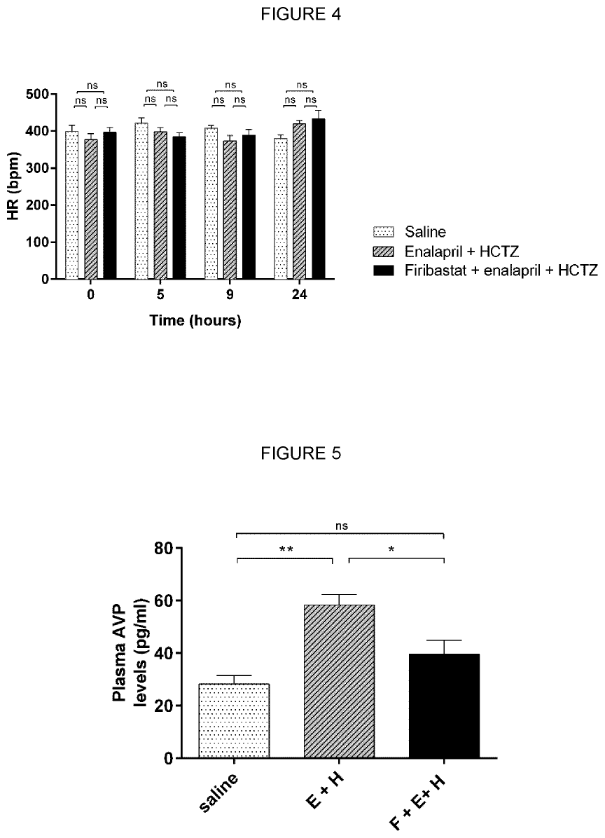

Firibastat (30 mg/kg) administered alone induced a significant decrease in BP (−35.4±5.2 mmHg) whereas enalapril (10 mg/kg) or HCTZ (10 mg/kg) given alone did not induce any significant change in BP decrease in DOCA-salt rats (FIG. 1). Dual combinations of enalapril (5 mg/kg) plus HCTZ (5 mg/kg), firibastat (30 mg/kg) plus enalapril (10 mg/kg) or firibastat (30 mg/kg) plus HCTZ (10 mg/kg) significantly decreased arterial BP by 36.9±4.4 mmHg, 11.6±3.7 mmHg and 30.1±9.9 mmHg respectively (FIG. 1).

Concomitant oral administration of firibastat (30 mg/kg) plus enalapril (5 mg/kg) plus HCTZ (5 mg/kg) significantly and markedly decreased MABP (FIG. 1) without significantly altering HR in conscious DOCA-salt rats (FIG. 2). A maximal decrease in MABP (−63.3±9.1 mmHg) was observed 5 hours after administration. The BP decrease induced by the triple combination of firibastat plus enalapril plus HCTZ was significantly different from that induced by each compound administered alone. Moreover, The BP decrease induced by the combination of firibastat plus enalapril plus HCTZ was significantly different from decreases induced by all the other dual combinations (firibastat plus enalapril, firibastat plus HCTZ and enalapril plus HCTZ).

In conclusion, combination of firibastat (30 mg/kg) plus enalapril (5 mg/kg) plus HCTZ (5 mg/kg) potentiated the BP decrease induced by firibastat (30 mg/kg), even more that the dual combination enalapril (5 mg/kg) plus HCTZ (5 mg/kg). These surprising results suggest that the existence of a synergy of action between firibastat, enalapril and HCTZ in the DOCA-salt model, by blocking respectively the brain and the systemic RAS activities, and increasing diuresis, leading to a profound BP decrease.

### Effects of 9-Day Chronic Oral Administration of Firibastat, Enalapril and HCTZ in Combination on BP and HR in Freely Moving DOCA-Salt Rats

We studied in alert DOCA-salt rats the effects on BP and HR of oral daily 9-day chronic administration of the dual combination of enalapril (5 mg/kg) plus HCTZ (5 mg/kg) or the triple combination of firibastat (30 mg/kg) plus enalapril (5 mg/kg) plus HCTZ (5 mg/kg). On day 9, we studied the time course of the effects of oral administration of these combinations on BP during 24 hours in alert DOCA-salt rats.

After 5 and 9 hours, concomitant oral administration of enalapril (5 mg/kg) plus HCTZ (5 mg/kg) or firibastat (30 mg/kg) plus enalapril (5 mg/kg) plus HCTZ (5 mg/kg) significantly and markedly decreased MABP (FIG. 3) without significantly altering HR in conscious DOCA-salt rats (FIG. 4). The decrease in MABP induced by the triple combination of firibastat (30 mg/kg) plus enalapril (5 mg/kg) plus HCTZ (5 mg/kg) was maximal 5 hours after administration and persisted after 9 hours, with significant decreases in MABP of 61.9±6.2 mmHg and 49.3±7.4 mmHg (P<0.0001), respectively (FIG. 3). After 24 hours, no decrease on BP was observed.

Five hours after administration, the BP decrease induced by the triple combination of firibastat (30 mg/kg) plus enalapril (5 mg/kg) plus HCTZ (5 mg/kg) was significantly different from that induced by the dual combination of enalapril (5 mg/kg) plus HCTZ (5 mg/kg) (61.9±6.2 mmHg and 31.3±8.2 mmHg (P<0.05), respectively).

As in acute treatment, 9-day chronic treatment with the triple combination of firibastat (30 mg/kg/day) plus enalapril (5 mg/kg/day) plus HCTZ (5 mg/kg/day) potentiated the BP decrease induced by firibastat (30 mg/kg/day). The synergy of action was even more marked when comparing the effects to the dual combination of enalapril (5 mg/kg/day) plus HCTZ (5 mg/kg/day). These results demonstrate also the absence of tolerance to the antihypertensive effect of triple combination of firibastat (30 mg/kg/day) plus enalapril (5 mg/kg/day) plus HCTZ (5 mg/kg/day) after repeated administrations.

Overall, these data indicate the existence, even after repeated administration, of a synergy of action between firibastat, enalapril and HCTZ to regulate BP in hypertensive DOCA-salt rats.

Blocking together brain RAS hyperactivity, systemic RAS activity and increasing diuresis with a triple combination of firibastat, enalapril and HCTZ represents a novel and original therapeutic treatment of HTN enabling further BP decrease in in hypertensive patients, more specifically on difficult-to-treat and resistant hypertensive patients.

### Effect of 10-Day Chronic Oral Administration of Firibastat, Enalapril and HCTZ in Combination on Plasma Arginine-Vasopressin (AVP) Levels in Conscious Hypertensive DOCA-Salt Rats

On day 10, levels of plasma AVP (known as the anti-diuretic hormone) in DOCA-salt rats which received chronic oral saline treatment were 28.2±3.3 pg/mL. The plasma AVP levels in DOCA-salt rats, 5 hours after repeated daily oral administrations of the dual combination of enalapril (5 mg/kg/day) plus HCTZ (5 mg/kg/day) or the triple combination of firibastat (30 mg/kg/day) plus enalapril (5 mg/kg/day) plus HCTZ (5 mg/kg/day) were increased by 107% and 40% (58.3±4.0 μg/mL and 39.6±5.3 μg/mL vs 28.2±3.3 μg/mL, respectively) when compared to DOCA-salt rats receiving chronic saline (FIG. 5).

The difference in plasma AVP levels between DOCA-salt rats receiving chronic treatment with the dual combination of enalapril (5 mg/kg/day) plus HCTZ (5 mg/kg/day) and DOCA-salt rats receiving chronic treatment with the triple combination of firibastat (30 mg/kg/day) plus enalapril (5 mg/kg/day) plus HCTZ (5 mg/kg/day) was 18.8 μg/mL. The addition of firibastat to the dual combination of enalapril and HCTZ reduced by 62% the increase in plasma AVP levels observed in DOCA-salt rats that received this dual combination (One-way ANOVA followed by Tukey's test, P<0.05).

1-21. (canceled)

22. A method of treating arterial hypertension or indirectly or heart failure comprising administering a pharmaceutical combination comprising (i) firibastat, (ii) a diuretic and (iii) a blocker of the systemic renin-angiotensin system selected from the group consisting of angiotensin I converting enzyme inhibitors and angiotensin II receptor type 1 antagonists to a subject in need of treatment.

23. The method according to claim 22, wherein the diuretic is selected from the group consisting of chlorothiazide, hydrochlorothiazide, chlorthalidone, indapamide, furosemide, torsemide, amiloride, triamterene, spironolactone and eplerenone.

24. The method according to claim 23, wherein the diuretic is hydrochorothiazide.

25. The method according to claim 22, wherein the blocker of the systemic renin-angiotensin system is selected from the group of angiotensin converting enzyme inhibitors consisting of lisinopril, enalapril, quinapril, ramipril, benazepril, captopril, cilazapril, fosinopril, imidapril, moexipril, trandolapril, or perindopril.

26. The method according to claim 22, wherein the blocker of the systemic renin-angiotensin system is enalapril.

27. The method according to claim 22, wherein the blocker of the systemic renin-angiotensin system is selected from the group consisting of angiotensin II receptor type 1 antagonists consisting of losartan, candesartan, irbesartan, telmisartan, valsartan, olmesartan, eprosartan and azilsartan.

28. The method according to claim 22, wherein the blocker of the systemic renin-angiotensin system is valsartan.

29. The method according to claim 22, wherein the diuretic is hydrochlorothiazide and the blocker of the systemic renin-angiotensin system is enalapril.

30. The method according to claim 22, wherein the diuretic is hydrochlorothiazide and the blocker of the systemic renin-angiotensin system is valsartan.

31. The method according to claim 22, wherein the diuretic is indapamide and the blocker of the systemic renin-angiotensin system is perindopril.

32. The method according to claim 22, wherein the diuretic is chlorthalidone and the blocker of the systemic renin-angiotensin system is azilsartan.

33. The method according to claim 29, containing firibastat in an amount in the range from 100 to 1500 mg, hydrochlorothiazide in an amount in the range from 5 to 30 mg, and enalapril in an amount in the range from 2.5 to 50 mg.

34. The method according to claim 29, containing firibastat in an amount in the range from 300 to 600 mg, hydrochlorothiazide in an amount in the range from 5 to 15 mg, and enalapril in an amount in the range from 2.5 to 15 mg.

35. The method according to claim 22, wherein the three active ingredients are administered simultaneously or sequentially.

36. A kit of parts comprising a pharmaceutical combination containing (i) firibastat, (ii) a diuretic, (iii) a blocker of the systemic renin-angiotensin system selected from the group consisting of angiotensin I converting enzyme inhibitors and angiotensin II receptor type 1 antagonists, in the form of one, two or three separate units of the components (i) to (iii), for a simultaneous or sequential administration.

37. The kit according to claim 36, wherein (ii) the diuretic is selected from the group consisting of hydrochorothiazide, indapamide, amiloride and chlorthalidone, and (iii) the blocker of the systemic renin-angiotensin system is selected from the group consisting of angiotensin I converting enzyme inhibitors consisting of enalapril, perindopril, ramipril and benazepril or from angiotensin II receptor type 1 antagonists the group consisting of losartan, valsartan, candesartan, irbesartan and azilsartan.

38. The kit according to claim 36, wherein (ii) the diuretic is hydrochlorothiazide and (iii) the blocker of the systemic renin-angiotensin system is enalapril.

39. The kit according to claim 36, wherein (ii) the diuretic is hydrochlorothiazide and (iii) the blocker of the systemic renin-angiotensin system is valsartan.

40. The kit according to claim 36, wherein (ii) the diuretic is indapamide and (iii) the blocker of the systemic renin-angiotensin system is perindopril.

41. The kit according to claim 36, wherein (ii) the diuretic is chlorthalidone and (iii) the blocker of the systemic renin-angiotensin system is azilsartan.
